(19) United States
(12) Patent Application Publication
(10) Pub. No.: US 2013/0150701 A1
(43) Pub. Date: Jun. 13, 2013

(54) MULTI-LUMEN THORACIC CATHETER AND USES THEREOF

(71) Applicants: Sara Budar, Honolulu, HI (US); Zachary Wayne Carr, Carmel, IN (US); Lauren Jennifer Griggs, Hillsborough, NJ (US); Gerald Riccardello, Succasunna, NJ (US); Stephanie Spelman, Randolph, NJ (US); Vikki Hazelwood, Wayne, NJ (US); David B. Pearlstone, Easton, CT (US); Arthur Ritter, Morristown, NJ (US)

(72) Inventors: Sara Budar, Honolulu, HI (US); Zachary Wayne Carr, Carmel, IN (US); Lauren Jennifer Griggs, Hillsborough, NJ (US); Gerald Riccardello, Succasunna, NJ (US); Stephanie Spelman, Randolph, NJ (US); Vikki Hazelwood, Wayne, NJ (US); David B. Pearlstone, Easton, CT (US); Arthur Ritter, Morristown, NJ (US)

(21) Appl. No.: 13/669,868

(22) Filed: Nov. 6, 2012

Related U.S. Application Data
(60) Provisional application No. 61/557,276, filed on Nov. 8, 2011.

(51) Int. Cl. A61M 1/00; A61M 5/14; A61B 5/00; A61B 10/04; A61B 5/01

(52) U.S. Cl. CPC A61M 1/008; A61B 10/04; A61B 5/01; A61B 5/0059; A61M 5/14
USPC 600/407; 604/43; 604/28; 600/562; 600/549

(57) ABSTRACT

A multi-lumen catheter for use in a cavity having a main lumen surrounded by a wall, and at least one access lumen positioned in or on the wall, the one access lumen conveys a solution to the cavity and said main lumen. A method for treating or preventing fluid or accumulation in a body cavity including (a) aseptically inserting through an incision at an insertion site the catheter comprising a main drainage lumen surrounded by a wall and the one access lumen positioned in or on the wall, (b) securing the inserted catheter by closing the incision with a suture, (c) infusing a physiological solution through the one access lumen to dilute a drainage fluid, (d) connecting a distal end of a main drainage lumen of the catheter to a suction drainage system, and (e) applying a vacuum force to the suction drainage system to remove the diluted drainage fluid.

### CROSS-REFERENCE TO RELATED APPLICATION

This application is a Section 111(a) application relating to and claims the benefit of commonly owned, co-pending U.S. Provisional Application Ser. No. 61/557,276 entitled “MULTI-LUMEN THORACIC CATHETER AND USES THEREOF”, filed Nov. 8, 2011, the entirety of which is incorporated herein by reference.

### FIELD OF THE INVENTION

The present invention relates to a multi-lumen thoracic catheter and its use for delivering therapeutic agents into or diagnosing a disease in a body cavity.

### BACKGROUND OF THE INVENTION

1. Pathologic Conditions that Require Use of Thoracic Catheters

In the human body the thoracic cavity, a hollow cavity, which is enclosed by the ribs, vertebral column, and the sternum and is separated from the abdominal cavity by the diaphragm, contains the lungs, the middle and lower airways, the heart, and various major blood vessels. Two thin membranes, known as pleurae, line the border of the cavity. Each pleura is continuous with two sides, the visceral pleura which covers the surface of a lung and the parietal pleura which is attached to the chest wall and diaphragm. These visceral and parietal pleurae meet at the hilum of each lung. The inter membrane space between each pleura creates a slit-like cavity, known as the pleural cavity. The pleurae produce a serous fluid, known as pleural fluid, which fills the pleural cavity a few millimeters thick. FIG. 10 depicts a transverse slice of the thoracic cavity, where the various structures discussed can be visualized.

The pleural cavity and pleural fluid play important roles in respiration. First, the fluid acts as a lubricant which allows the visceral and parietal pleurae to glide easily past each other with the movements of breathing. Second, the surface tension of the pleural fluid creates a strong adhesive force between the parietal and visceral pleurae, binding the pleurae together. This strong adhesive force is vital in keeping the lungs inflated as there are several opposing forces acting on the lungs. Both the natural elasticity of the alveoli and the surface tension of the alveolar fluid act as a recoil force, forcing the lungs to contract, while the elasticity of the chest wall acts to expand the thorax and force it outward. Since the pleural fluid binds the lungs to the chest wall, forces on the lungs are opposed by the force on the chest wall. This results in a negative pleural pressure (relative to the atmosphere), which causes the lungs to remain inflated. Not only does the surface tension ensure inflation, but it is also responsible for the lungs' ability to passively change volume during respiration. As the chest wall and diaphragm expand, the pleural fluid transmits these movements to the lungs via a pressure gradient between two sides of the pleura.

Transpulmonary pressure, the difference between the alveolar pressure and the pleural fluid pressure, determines the size of the lungs at any given time. Therefore, it is important that a negative pressure is maintained in the pleural space at all times. Any condition that equalizes or creates a positive pleural pressure causes immediate lung collapse. The amount of pleural fluid in the pleural space must therefore remain at a minimal level.

Pleural fluid is continuously released into the pleural cavity, and is continuously reabsorbed by the lymphatic system. The lymphatic system can reabsorb at up to 40 times the normal rate, if necessary, due to abnormal fluid accumulation. However, if fluid accumulates at a rate greater than the lymphatic system can reabsorb, the pleural pressure becomes positive resulting in immediate lung collapse. When a lung collapses, the lung is unable to expand during respiration leading to difficulty breathing and hypoxemia, a lack of oxygen in the blood (Marieb, E. and Hoehn, K., Human Anatomy and Physiology. 8th ed. San Francisco: Benjamin-Cummings Pub, 2010).

### 2. Clinical Practice

Medical indications for surgical thoracic catheter insertion include, but are not limited to: (1) drainage of hemothorax, or large pleural effusion of any cause; (2) drainage of large pneumothorax (greater than 25%); (3) prophylactic placement of a thoracic catheter in a patient with suspected chest trauma before transport to specialized trauma center; (4) flail chest segment requiring ventilator support; (5) severe pulmonary contusion with effusion; and (6) evacuation (and maintenance of evacuation of pleural space) following thoracotomy (surgery in which the pleural space is purposely opened) (“CHEST TUBE INSERTION.” APPS. Web. Oct. 29, 2010. at apps.med.buffalo.edu/procedures/chesttube.asp?p=7, the entire contents of which are incorporated herein by reference in their entirety).

Insertion of a thoracic catheter is usually accomplished using a scalpel and a Kelly clamp or a trocar. For both methods, the point of insertion in the chest most commonly occurs on the side (lateral thorax), at a line drawn from the armpit (anterior auxiliary line) to the side (lateral) of the nipple in males, or to the side (about 2 inches) above the sternoxiphoid junction (lower junction of the sternum, or chest bone) in females. The skin is sterilized with antiseptic solution covering a wide area, and local anesthesia is administered to minimize discomfort. At the rib chosen for insertion, the skin over the rib is anesthetized with an anesthetic, such as lidocaine. The patient's arm is placed over the head with a restraint on the affected side. An incision is made, using a scalpel, through the skin, muscle tissue and into the pleura, and a Kelly clamp is used to open the pleural cavity. The tube is inserted into the pleural space and the clamp is removed. The tube is then manually advanced. For trocar insertion, the tube and trocar are slowing guided through the hole in the pleura into the pleural space. The trocar is then removed and the tube is manually advanced. A silk suture is used to hold the tube firmly in place and the tube is attached to a suction drain system. An x-ray is taken to visualize the status of the tube placement (“Chest Tube Insertion—Procedure, Recovery, Blood, Removal, Pain, Complications, Infection, Heart, Cells, Children, Cancer, Definition, Purpose, Demographics, Description, Diagnosis/Preparation, Aftercare.” Encyclopedia of Surgery: A Guide for Patients and Caregivers. Web. Oct. 29, 2010, at surgeryencyclopedia.com/Ce-Fi/Chest-Tube-Insertion.html, the entire contents of which are incorporated by reference herein in their entirety). A thoracic catheter may be inserted intra-operatively or at the bedside. For intra-operative placement, an incision is made in the same location as described, but without either positioning the patient or using local anesthetic.

The suction drainage system often consists of a three bottle system. The first chamber is the collection chamber. This chamber attaches directly to the thoracic catheter in the patient. Its function is to collect the drained fluid allowing for visualization and recording of the fluid. The second chamber is a water seal chamber. The water seal acts as a one way valve, which prevents air and fluid from returning to the pleural space during inspiration, but otherwise allows it to exit. On new drain system models, the water seal has recently been replaced by a mechanical one way valve. The third chamber is the suction control chamber, which controls the amount of suction allowed by the system. (“Chest Tube Insertion—Procedure, Recovery, Blood, Removal, Pain, Complications, Infection, Heart, Cells, Children, Cancer, Definition, Purpose, Demographics, Description, Diagnosis/Preparation, Aftercare.” Encyclopedia of Surgery: A Guide for Patients and Caregivers. Web. Oct. 29, 2010, at surgeryencyclopedia.com/Ce-Fi/Chest-Tube-Insertion.html, the entire contents of which are incorporated by reference herein in their entirety).

It is important to maintain a closed negative pressure system for two reasons: a negative pressure in the pleural space is necessary to prevent lung collapse and it is necessary to keep the loop closed to prevent pathogens from entering the body.

Thoracic catheters are available in various sizes, with manufacturers providing numerous prepackaged kits for thoracic catheter placement. The following factors are balanced in selecting a correct size for a thoracic catheter: the flow rate of the fluid that can be accommodated by the tube, the size of the patient, and the potential for clogging. The following is a table of suggested thoracic catheter size (“CHEST TUBE INSERTION.” APPS. Web. Oct. 29, 2010. at apps.med.buffalo.edu/procedures/chesttube, the contents of which are incorporated by reference herein in their entirety).

TABLE 1
Suggested Catheter Sizes

| RECIPEINT | SIZE OF TUBE |
|---|---|
| Adult or Teen Male | 28-32 Fr |
| Adult or Teen Female | 28 Fr |
| Child | 18 Fr |
| Newborn | 12-14 Fr |

Although these are suggested tube sizes, surgeons may opt to use larger bore tubes, from 32 to 40 Fr (French Size (medical tubing unit of measurement)), to ensure patency of the tube, i.e., the overall range of bore sizes is from 12 Fr to 40 Fr. Tubes may be cut at the distal end to adjust the length of the tube.

The thoracic catheter remains in place until imaging studies reveal that the underlying cause of the problem has been resolved and the pleural fluid is back at its normal volume (“CHEST TUBE INSERTION.” APPS. Web. Oct. 29, 2010. at apps.med.buffalo.edu/procedures/chesttube, the contents of which are incorporated by reference herein in their entirety).

### 3. Complications

Several complications can develop with thoracic catheter insertion. A major and common complication is occlusion. Thoracic catheters often become partially or completely occluded with blood clots and other fibrous material. These occlusions can lead to life-threatening complications, including tension pneumothorax and sepsis, and may require additional surgery if the resulting conditions cause loss of lung volume. A survey revealed 106 out of 106 (100%) surgeons surveyed had observed thoracic catheter clogging, and 93 of the 106 (87%) reported adverse patient outcomes from a clogged tube (Shanaz, S. et al., “Chest Tube Selection in Cardiac and Thoracic Surgery: A Survey of Chest Tube Related Complications and Their Management.” Wiley Periodicals 503-09, 2009).

Several techniques can be tried to dislodge the occluding material, but each method has significant drawbacks and often does not succeed in removing the material. One procedure is milking or stripping the thoracic catheter to attempt to manually dislodge the clot, but research has shown this can produce extremely high negative pressures in the thorax which are harmful to the patient. In certain cases, the surgeon may disconnect the suction in order to create tube patency. This method creates a pneumothorax and is a severe sterility issue. For these reasons, surgeons typically use large bore tubes to ensure patency, but even these tubes frequently become clogged (Shanaz, S. et al., “The Active Tube Clearance System,” International Society for Minimally Invasive Cardiothoracic Surgery 1st ser. 5.1, 2010).

The use of large bore tubes raises another issue related to thoracic catheter insertion, as large tubes are associated with significant patient discomfort (Shanaz, S. et al., “The Active Tube Clearance System,” International Society for Minimally Invasive Cardiothoracic Surgery 1st ser. 5.1, 2010). The thoracic catheter remains in the patient until the underlying cause is resolved, which can be an extended period of time. For this time, the tube is constantly in contact with the open skin wound, the ribs, and parietal pleura, which is highly sensitive to pain. Any size, but especially large bore, thoracic catheters irritate these areas causing patient discomfort, which can lead to other complications. An uncooperative patient may move or turn, resulting in a dislodged tube or a broken seal, leading to reinsertion of the tube and sterility issues. The only current methods for reducing pain are the use of smaller bore tubes and anesthetic or pain medication.

Another complication which can arise is a local or generalized infection from the procedure. The infection can attack either the wound site or cause empyema, a collection of pus within the cavity. Oral antibiotics can be administered to treat such infections, but if patient discomfort and tube occlusion can be reduced, the frequency of infection can be greatly reduced.

Clogging, pain and infection are three complications which frequently arise from the use of thoracic catheters. However, the solutions currently offered and the ones being developed do not meet the needs of the patients and surgeons.

The described invention provides a newly designed multi-lumen thoracic catheter, which incorporates the common features of existing catheters with an original design that can meet and exceed the needs of all parties.

The multi-lumen catheter of the present invention provides facilities for medical practitioners (e.g., surgeons and physicians) to overcome many of the short comings associated with the use of conventional thoracic catheters (i.e. the dangerous build-up of osculation's in the drainage tube, and pain inflicted on the patient by the drainage tube itself). As will be described hereinafter, the multi-lumen catheter provides medical practitioners with a means through which they can access spaces (e.g., the pleural cavity) that had been previously inaccessible by the use of conventional thoracic catheters, as shown in FIGS. 11A and 11B. Conventional thoracic catheters are used in a closed negative pressure system with a suction drainage device. This means that a seal must be maintained between the atmosphere and the skin that contacts the catheter in order to support respiratory functioning, and prevent infection. Because of this, surgeons are not able to access the pleural space or the interior of the main drainage lumen of the thoracic catheter without breaking the seal. This causes a problem when complications arise. The major complications, including occlusion and pain, occur within the regions that are inaccessible by conventional thoracic catheters (see FIG. 11A). The multi-lumen catheter providing access to the pleural space and the interior of the main drainage lumen, giving physicians the ability to access the areas where complications arise and allowing them to actively provide solutions to the underlying causes. The multi-lumen catheter allows access to the aforementioned inaccessible regions and the methods which use this access to provide solutions, for example occlusion and pain (see FIG. 11B).

### SUMMARY OF THE INVENTION

According to one aspect, the described invention provides a multi-lumen catheter for use in a cavity, comprising a main lumen surrounded by a wall; and at least one access lumen positioned in or on the wall, wherein said at least one access lumen conveys a solution to the cavity and said main lumen.

According to another aspect, the described invention provides a method for treating or preventing fluid accumulation or air accumulation in a body cavity of a subject using the multi-lumen catheter, the method comprising: (a) aseptically inserting through an incision at an insertion site of the subject the multi-lumen catheter comprising a main drainage lumen surrounded by a wall and at least one access lumen positioned in or on the wall; (b) securing the inserted multi-lumen catheter by closing the incision with a suture; (c) infusing a physiological solution through at least one access lumen of the multi-lumen catheter to dilute a drainage fluid; (d) connecting a distal end of a main drainage lumen of the multi-lumen catheter to a suction drainage system; and (e) applying a vacuum force to the suction drainage system to remove the diluted drainage fluid.

According to one embodiment of the method, the body cavity is a pleural cavity. According to another embodiment, the body cavity is a cranial cavity. According to another embodiment, the body cavity is a spinal cavity. According to another embodiment, the body cavity is a abdominal cavity. According to another embodiment, the body cavity is a pelvic cavity. According to another embodiment, the physiological solution in step (c) comprises a saline solution, Ringer's solution, 5% dextrose in water (D5W), or a mixture thereof. According to another embodiment, a thrombolytic agent is infused through an access lumen that exits into the main drainage lumen. According to another embodiment, the suction drainage system is a single-flow drainage system that only allows one direction of flow. According to another embodiment, the suction drainage system comprises a collection chamber, a water seal chamber, and a suction control chamber, wherein the collection chamber attaches the multi-lumen thoracic catheter to the subject; wherein the water seal chamber prevents air and fluid from returning to the pleural space; during inspiration; and wherein the suction control chamber controls the amount of suction allowed by the suction drainage system. According to another embodiment, the fluid accumulation or air accumulation in the pleural cavity of the subject results from a condition comprising pneumothorax, pleural effusion, chylothorax, empyema, hemothorax, hydrothorax, or a combination thereof. According to another embodiment, the fluid accumulation or air accumulation in the pleural cavity of the subject results from a condition selected from the group consisting of a pulmonary disease, a lung infection, a lung cancer, a breast cancer, and a surgery that affects a negative pressure in the pleural space. According to another embodiment, the insertion site is determined by reviewing clinical signs and chest imaging of the subject. According to another embodiment, the chest imaging comprises chest X-ray, chest fluoroscopy, computed tomography (CT), high-resolution computed tomography (CT), helical (spiral) computed tomography (CT), computed tomography (CT) angiography, magnetic resonance imaging (MRI), or ultrasonography. According to another embodiment, the insertion site is a lateral thorax, at a line drawn from an armpit to the nipple in male or to the side above the sternoxiphoid junction (lower junction of the sternum, or chest bone) in female. According to another embodiment, a size of the incision for the insertion of the multi-lumen catheter is similar to the diameter of the multi-lumen thoracic catheter being inserted. According to another embodiment, the method further comprises infusing a therapeutic agent through a second access lumen of the multi-lumen catheter. According to another embodiment, the therapeutic agent is a local anesthetic agent, and wherein the local anesthetic agent decreases pain associated with tissue irritation or tube insertion. According to another embodiment, wherein the local anesthetic is selected from the group consisting of benzocaine, lidocaine, and marcaine. According to another embodiment, the therapeutic agent is an anti-coagulant agent. According to another embodiment, infusing is performed as a bolus (single) infusion. According to another embodiment, infusing is performed as continuous infusion. According to another embodiment, the therapeutic agent is infused at a flow rate ranging from about 1 cc per hour to about 500 cc per hour. According to another embodiment, the therapeutic agent is an anti-infective agent comprising an antibiotic agent, an anti-tuberculin agent, an anti-fungal agent, or antiviral agent, wherein the anti-infective agent treats or prevents a localized infection. According to another embodiment, the therapeutic agent is anti-fungal agent. According to another embodiment, the therapeutic agent is anti-tuberculin agent. According to another embodiment, the therapeutic agent is a sclerotic agent, wherein the sclerotic agent induces adhesion between the parietal and visceral pleura. According to another embodiment, the sclerotic agent is infused as a bolus injection, and wherein the vacuum force is discontinued for an hour. According to another embodiment, the therapeutic agent is an anti-inflammatory agent, and wherein the anti-inflammatory agent decreases inflammation in the pleural space. According to another embodiment, the therapeutic agent is a thrombolytic agent, and wherein the thrombolytic agent dissolves clotted blood in the pleural space.

According to another aspect, the described invention provides a method for examining a tissue in a body cavity of a subject using the multi-lumen catheter, the method comprising: (a) aseptically inserting through an incision at an insertion site of the subject the multi-lumen catheter comprising a main drainage lumen surrounded by a wall and at least one access lumen positioned in or on the wall; (b) securing the inserted multi-lumen catheter by closing the incision with a suture; (c) inserting an endoscope through an access lumen of the multi-lumen catheter; and (d) examining the tissue in the body cavity of the subject.

According to one embodiment of the method, the body cavity is a pleural cavity. According to another embodiment, the body cavity is a cranial cavity. According to another embodiment, the body cavity is a spinal cavity. According to another embodiment, the body cavity is a abdominal cavity. According to another embodiment, the body cavity is a pelvic cavity. According to another embodiment, the method further comprises sampling a tissue in the pleural space, wherein the endoscope comprises endoscopic forceps for tissue biopsy. According to another embodiment, the method further comprises sampling a tissue in the pleural space, wherein a flexible biopsy forceps that is not incorporated into an endoscope is guided into the body cavity under fluoroscopy or blindly.

According to another aspect, the described invention provides a method for monitoring a physical or biochemical state of a tissue within a body cavity using the multi-lumen catheter, the method comprising: (a) aseptically inserting through an incision at an insertion site the multi-lumen catheter comprising a main drainage lumen surrounded by a wall and at least one access lumen positioned in or on the wall; (b) securing the inserted multi-lumen catheter by closing the incision with a suture; (c) introducing an instrument that measures the physical or biochemical state of the tissue within the body cavity through at least one access lumen of the multi-lumen catheter; and (d) monitoring the physical or biochemical state of the tissue within the body cavity, wherein the physical or biochemical state comprises an electrical parameter, a thermal parameter, a photoelectric parameter, a barometric parameter, or a combination thereof.

According to one embodiment of the method, the body cavity is a pleural cavity. According to another embodiment, the body cavity is a cranial cavity. According to another embodiment, the body cavity is a spinal cavity. According to another embodiment, the body cavity is a abdominal cavity. According to another embodiment, the body cavity is a pelvic cavity.

### BRIEF DESCRIPTION OF THE DRAWINGS

For a more complete understanding of the present invention, reference is made to the following detailed description of an embodiment considered in conjunction with the accompanying drawings, in which:

FIG. 1 is a perspective view of a multi-lumen thoracic catheter which is constructed in accordance with one embodiment of the present invention;

FIGS. 2 and 3 are partial view of the multi-lumen thoracic catheter shown in FIG. 1;

FIG. 4A is a perspective views of a multi-lumen thoracic catheter which is constructed in accordance with another embodiment of the present invention;

FIG. 4B is a partial view of the multi-lumen thoracic catheter shown in FIG. 4A;

FIGS. 5A and 5B are partial views of a multi-lumen thoracic catheter which is constructed in accordance with another embodiment of the present invention;

FIG. 6A is a perspective view of a multi-lumen thoracic catheter which is constructed in accordance with another embodiment of the present invention;

FIG. 6B is a partial view of the multi-lumen thoracic catheter shown in FIG. 6A;

FIG. 7 is a perspective view of a multi-lumen thoracic catheter which is constructed in accordance with another embodiment of the present invention;

FIG. 8 is a partial view of the multi-lumen thoracic catheter shown in FIG. 7;

FIG. 9 is a cross-sectional view of the multi-lumen thoracic catheter shown in FIG. 7;

FIG. 10 shows traverse slice of thoracic cavity;

FIGS. 11A and 11B show diagrams of accessible areas in the lung;

FIG. 12 shows the concentration of dye vs. time plot for injection, as well as a fluid injected through the access lumen can diffuse to the wound site and exist in significant concentrations relative to the rest of the pleural space; and

FIG. 13 shows the concentration of dye vs. time plot for infusion, as well as a fluid injected through the access lumen can diffuse to the wound site and exist in significant concentrations relative to the rest of the pleural space.

### DETAILED DESCRIPTION OF THE INVENTION

### 1. Glossary

The term “administering” as used herein refers to giving or applying. The term “administering” includes in vivo administration, as well as administration directly to tissue ex vivo.

The term “anti-coagulant” as used herein refers to a substance that inhibits blood coagulation (blood clotting). Examples of anti-coagulants suitable in the context of the present invention include, but are not limited to, warfarin, coumadin, acenocoumarol, phenprocoumon, phenindione, and heparin.

The term “aseptic” and its various grammatical forms, as used herein, refers to a state free from living pathogenic organisms or methods used to protect against infection by pathogenic microorganism.

The term “antiseptic solution” as used herein refers to a substance that inhibits the growth and development of microorganisms. Antiseptics are a diverse class of drugs, which are applied to skin surfaces or mucous membranes for their anti-infective effects. These may be either bacteriocidal or bacteriostatic. Their uses include cleansing of skin and wound surfaces after injury, preparation of skin surfaces prior to injections or surgical procedures, and routine disinfection of the oral cavity as part of a program of oral hygiene. Suitable antiseptics for skin cleaning, for example, include, but are not limited to, benzalkonium chloride, chlorhexidine, hexachlorophine, iodine compounds, mercury compounds, alcohol, and hydrogen peroxide.

The term “anesthetic” as used herein refers to an agent that causes loss of sensation in a human or other mammal with or without the loss of consciousness.

The term “local anesthetic” as used herein refers to an anesthetic agent that induces insensitivity to pain pertaining to or affecting a particular part or area of the body without concomitant loss of consciousness by reversibly inhibiting peripheral nerve excitation and/or conduction. Local anesthetics suitable for use in the present invention, include, but are not limited to, ester-based anesthetics, and ester analogs of other anesthetics. Ester-based anesthetics include, but are not limited to, cocaine, procaine, chloroprocaine, tetracaine, benzocaine, amethocaine, chlorocaine, butamben, dibucaine, and the like. Amide-based anesthetics include, but are not limited to, lidocaine, prilocalne, mepivacaine, ropivocaine, etidocaine, levobupivacaine, bupivacaine, and the like. Other anesthetics suitable for use in the present invention include, but are not limited to, ester analogs of aconitine, dyclonine, ketamine, pramoxine, safrole, and salicyl alcohol. Such ester analogs can contain an ester group anywhere within the structure.

The term “anti-fungal agent” as used herein means any of a group of chemical substances having the capacity to inhibit the growth of (fungistatic) or to kill (fungicidal) fungi. Anti-fungal agents include, without limitation, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, PyrroInitrin, Siccanin, Tubercidin, Viridin, Butenafine, Naftifine, Terbinafine, Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “antibiotic agent” as used herein means any of a group of chemical substances having the capacity to inhibit the growth of (bacteriostatic), or to kill bacteria (bacteriocidal), and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefinetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives.

The term “anti-tuberculin agent” or “anti-mycobacterial agent” as used herein refers to any compound or mixture of compounds effective in inhibiting, attenuating, or combating a tuberculosis-causing or other disease-causing mycobacterium species. Examples of anti-tuberculin agents include, but are not limited to, isoniazid, activated isoniazid, rifampin, capreomycin, ethionamide, cycloserine, ciprofloxacin, amikacin, streptomycin, ethambutol, and pyrazinamide.

The term “anti-viral agent” as used herein means any of a group of chemical substances having the capacity to inhibit the replication of or to destroy viruses used chiefly in the treatment of viral diseases. Examples of anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT), and Xenazoic Acid.

When referring to humans, the body and its parts are always described using the assumption that the body is standing upright. Portions of the body which are closer to the head end are “superior” (corresponding to cranial in animals), while those farther away are “inferior” (corresponding to caudal in animals). Objects near the front of the body are referred to as “anterior” (corresponding to ventral in animals); those near the rear of the body are referred to as “posterior” (corresponding to dorsal in animals). A transverse, axial, or horizontal plane is an X-Y plane, parallel to the ground, which separates the superior/head from the inferior/feet. A coronal or frontal plane is an Y-Z plane, perpendicular to the ground, which separates the anterior from the posterior. A sagittal plane is an X-Z plane, perpendicular to the ground and to the coronal plane, which separates left from right. The midsagittal plane is the specific sagittal plane that is exactly in the middle of the body.

Structures near the midline are called medial and those near the sides of animals are called lateral. Therefore, medial structures are closer to the midsagittal plane, lateral structures are further from the midsagittal plane. Structures in the midline of the body are median.

Ipsilateral means on the same side, contralateral means on the other side and bilateral means on both sides. Structures that are close to the center of the body are proximal or central, while ones more distant are distal or peripheral. For example, the hands are at the distal end of the arms, while the shoulders are at the proximal ends.

The term “biomarkers” (or “biosignatures”) as used herein refers to peptides, proteins, nucleic acids, antibodies, genes, metabolites, or any other substances used as indicators of a biologic state. A biomarker is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “biopsy” as used herein refers to a procedure for removing a piece of tissue or a sample of cells from the body so that it can be analyzed in a laboratory. For example, during endoscopy, special tools are passed through the tube to take a small sample of tissue to be analyzed

The term “body cavity” as used herein refers to an inner or open space of a tissue or of a body organ. The two major body cavities are the dorsal cavity and the ventral cavity. The dorsal cavity includes the cranial and spinal (vertebral) cavities. The ventral cavity is larger than the dorsal cavity and has two portions separated by the muscular diaphragm. Superior to the diaphragm is the thoracic cavity, and inferior to the diaphragm is the larger abdominopelvic cavity, which contains abdominal and pelvic cavities. The portions of the thoracic cavity that contain the lungs are called the left and right pleural cavities and are on the body's left and right sides, respectively.

The term “cranial cavity” or “intercranial cavity” as used herein refers to the space or hollow within the skull.

The term “spinal cavity” or “vertebral cavity” as used herein refers to the opening that runs through the center of the column of spinal bones (vertebrae), and through which the spinal cord passes.

The term “abdominal cavity” as used herein refers to a body cavity that holds the bulk of the viscera and which is located inferior to the thoracic cavity, and above the pelvic cavity. Organs of the abdominal cavity include the stomach, liver, gallbladder, spleen, pancreas, urinary bladder, small intestine and large intestine. The abdominal cavity is lined with a protective membrane termed the peritoneum. The viscera are also covered, in the front, with a fatty layer called the omentum (or omental apron).

The term “pelvic cavity” as used herein refers to a body cavity that is bounded by the bones of the pelvis and which primarily contains reproductive organs, the urinary bladder, and the rectum.

The term “component” as used herein refers to a constituent part, element or ingredient.

The term “condition”, as used herein, refers to a variety of health states and is meant to include disorders or diseases caused by any underlying mechanism or disorder, injury, and the promotion of healthy tissues and organs.

The term “contact” and its various grammatical forms as used herein refers to a state or condition of touching or of immediate or local proximity. Contacting a composition to a target destination, such as, but not limited to, an organ, a tissue, a cell, or a tumor, may occur by any means of administration known to the skilled artisan.

The terms “disease” or “disorder” as used herein refer to an impairment of health or a condition of abnormal functioning.

The term “endoscopic forceps” as used herein refers to a device designed to be used for grasping or removal of foreign objects.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “infusing” as used herein refers to introducing a fluid or a solution into the body.

The term “incision” as used herein refers to a cut into a body tissue or organ, especially one made during surgery.

The term “Kelly clamp” as used herein refers to a curved hemostat without teeth.

As used herein the term “lumen” means a cavity or channel within a tubular structure.

The term “monitoring”, as used herein, refers to detecting, observing, predicting, analyzing or determining physical and biochemical state of the pleural tissue of a subject.

The term “pleural effusion” as used herein refers to an abnormal buildup of fluid between the layers of tissue that line the lungs and chest cavity. The body produces pleural fluid in small amounts to lubricate the surfaces of the pleura, the thin tissue that lines the chest cavity and surrounds the lungs. A pleural effusion is an abnormal, excessive collection of this fluid. There are two different types of effusions that can develop. Transudative pleural effusions generally are caused by fluid leaking into the pleural space. This is caused by increased pressure in, or low protein content in, the blood vessels. Congestive heart failure is the most common cause. Exudative effusions generally are caused by blood clots in the lung blood vessels (pulmonary emboli), infection, inflammation, lung injury, and drug reactions.

The term “pleural space” or “pleural cavity” as used herein refers to a small area between layers of the pleurae (the thin covering that protects and cushions the lungs). The pleural space is normally filled with a small amount of fluid.

The term “prevent” as used herein refers to the keeping, hindering or averting of an event, act or action from happening, occurring, or arising.

The term “physiological solution” as used herein means any isotonic buffer solution, including, but not limited to, saline solution, Ringer's solution, phosphate-buffered saline solution, and 5% dextrose in water (D5W).

The term “reduced” or “to reduce” as used herein refer to a diminution, a decrease, an attenuation or abatement of the degree, intensity, extent, size, amount, density or number.

The term “safe triangle” as used herein refers to the triangle bordered by the anterior border of the latissimus dorsi, the lateral border of the pectoralis major muscle, a line superior to the horizontal level of the nipple, and an apex below the axilla.

The term “saline solution” as used herein refers to a solution containing approximately 0.9% sodium chloride solubilized in water (including deuterated water), such as distilled water, and which solutions contain substantially no other additives but may be pH adjusted with hydrochloric acid or sodium hydroxide.

The term “serous fluid” as used herein refers to a fluid that lies between the membrane lining the body cavities (parietal) and those covering the organs within the cavities.

The term “single flow drainage system” as used herein refers to a flow drainage system that only allows one direction of flow.

The terms “subject” or “individual” or “patient” are used interchangeably to refer to a member of an animal species of mammalian origin, including but not limited to, a mouse, a rat, a cat, a goat, sheep, horse, hamster, ferret, platypus, pig, a dog, a guinea pig, a rabbit and a primate, such as, for example, a monkey, ape, or human.

The term “suture” as used herein refers to any product used to close wounds or connect tissue. The term includes any strand of material used to ligate (tie) blood vessels or approximate tissues.

The term “therapeutic agent” as used herein refers to a drug, molecule, nucleic acid, protein, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably. The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutically effective amount” or an “amount effective” of one or more of the active agents is an amount that is sufficient to provide the intended benefit of treatment. Dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a surgeon using standard methods.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

The term “thrombolytic agent” as used herein refers to a substance used to dissolve a clot (thrombus) and thereby reopen an artery or vein. All thrombolytic agents are serine proteases and convert plasminogen to plasmin, which breaks down the fibrinogen and fibrin and dissolves the clot. Examples of thrombolyic agents include, but are not limited to, reteplase (r-PA or Retavase), alteplase (t-PA or Activase), urokinase (Abbokinase), prourokinase, anisoylated purified streptokinase activator complex (APSAC), and streptokinase.

### 2. Multi-Lumen Thoracic Catheter

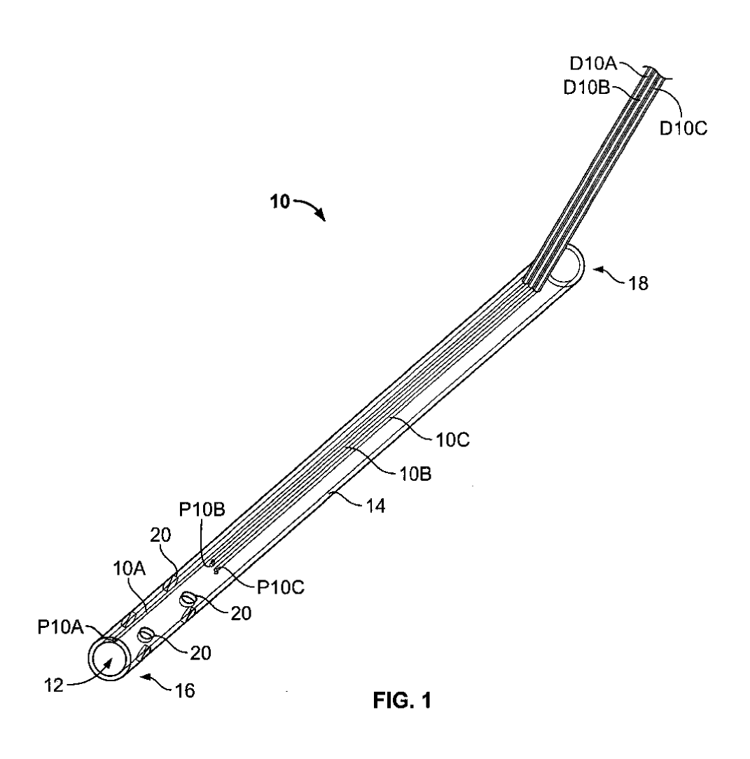

FIGS. 1-3 illustrate a multi-lumen thoracic catheter 10 (hereinafter “the catheter 10”) constructed in accordance with one embodiment of the present invention. The catheter 10 includes a main drainage lumen 12, which is surrounded by a wall 14. According to one embodiment, the wall 14 of the lumen 12 may be fashioned in accordance with the configuration of a conventional thoracic catheter such as a Teleflex 32 Fr 6 Eyed Soft PVC catheter, although it may be fashioned in accordance with other sized thoracic catheters (i.e., according to the size of the patient). The wall 14 has a proximal end 16 (i.e., closest to the cavity) and a distal end 18 (i.e., distant from the cavity) (See FIG. 3). A plurality of drainage eyes 20 are positioned in the wall 14, adjacent to the proximal end 16 of the lumen 12. According to one embodiment, radio-opaque markers (e.g., for x-ray imaging of exact location of all components of the thoracic catheter) and suture tabs may be included in the wall 14 of the lumen 12 (these elements are not shown in the Figures). According to one embodiment, the distal end 18 of the wall 14 is attachable to a suction drain vacuum device, such as a Pleur-Evac® Chest Drainage system (not shown in the Figures).

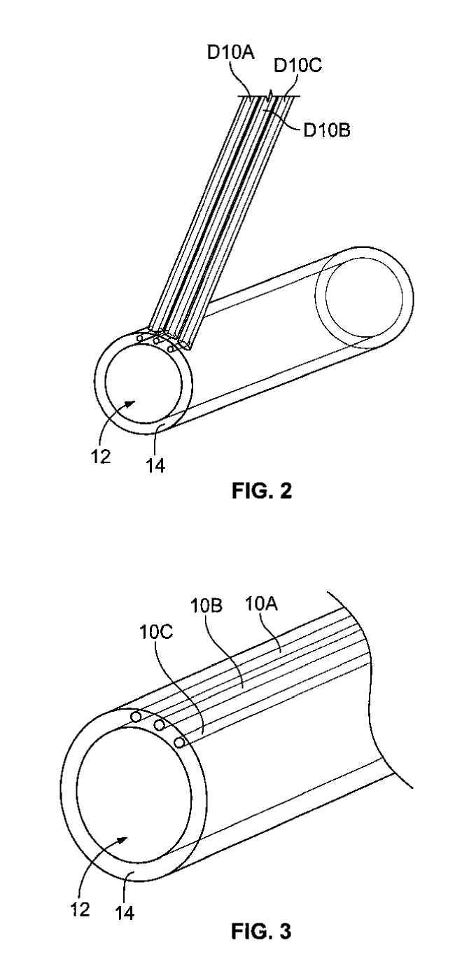

According to one embodiment, access lumens 10A, 10B, and 10C are positioned in the wall 14 of the lumen 12. More particularly, with specific reference to FIG. 2, the access lumens 10A, 10B, and 10C may be 20 gauge in diameter and enter the wall 14 adjacent to the distal end 18 of the wall 14. FIG. 2 illustrates the position where the access lumens 10A, 10B, and 10C enter the wall 14 of the lumen 12. It is understood that dimensions such as the diameter of the access lumens 10A, 10B, and 10C may vary, for instance, +150% (mean value). According to one embodiment, the diameter of the access lumens 10A, 10B, and 10C ranges from 10 to 16 Fr for pediatric catheters. According to another embodiment, the diameter of the access lumens 10A, 10B, and 10C ranges from 20 to 40 Fr (French Size (medical tubing unit of measurement)). The longitudinal axes of the access lumens 10A, 10B, and 10C are oriented parallel to the longitudinal axis of the lumen 12, as illustrated in FIG. 3.

The access lumens 10A, 10B, and 10C have proximal ends P10A, P10B, and P10C, respectively. The proximal ends P10A, P10B, and P10C may be positioned along the longitudinal axes of the walls 14 of the lumens 12, respectively, in a variety of positions. For example, they may terminate at i) the proximal end 16 of the wall 14 or ii) a position that is located adjacent to the proximal end 16 of the wall 14 but within the cavity of the patient. Also the outflow of the proximal ends P10A, P10B, and P10C of the access lumens 10A, 10B, and 10C, respectively, may be oriented in one of the following ways: i) in the longitudinal direction, ii) transversely inwardly into the lumen 12, or iii) transversely outwardly away from the lumen 12.

The access lumen 10A of the catheter 10 is positioned at the proximal end of the lumen 12, and its outflow is oriented in the longitudinal direction. The access lumen 10B is located adjacent to the proximal end of the lumen 12, and its outflow is oriented transversely outwardly away from the lumen 12. The access lumen 10C is located away from the proximal end of the lumen 12, and its outflow is oriented transversely inwardly into the lumen 12. These positions and orientations will vary according to various embodiments, as they are provided to enable the surgeon or physician to facilitate procedures that are directed at reducing various complications. The methods that are directed at reducing various complications are described in detail hereinbelow.

The access lumens 10A, 10B, and 10C have distal ends D10A, D10B, and D10C., respectively. According to one embodiment, the distal ends D10A, D10B, and D10C may be attached to luer-lock valves, to facilitate the injection or infusion of specific solutions, which are directed at reducing complications. Such procedures for operating the catheter 10 that are directed at reducing various complications are described in detail hereinbelow.

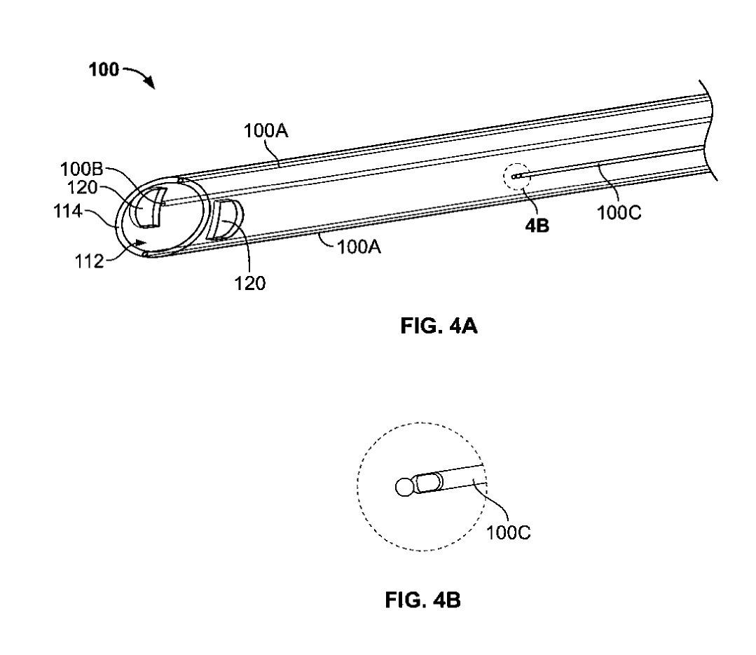

FIGS. 4A and 4B depict a second embodiment of the present invention. Elements illustrated in FIGS. 4A and 4B, which correspond, either identically or substantially, to the elements described above with respect to the embodiment of FIGS. 1-3 have been designated by corresponding reference numerals increased by one hundred. Unless otherwise stated, the embodiments of FIGS. 4A and 4B are constructed and assembled in the same basic manner as the embodiment of FIGS. 1-3.

FIGS. 4A and 4B illustrate a multi-lumen thoracic catheter 100 constructed in accordance with one embodiment of the present invention. The catheter 100 includes a main drainage lumen 112 which is surrounded by a wall 114. According to one embodiment, access lumens 100A, 100B, and 100C are positioned in the wall 114 of the lumen 112.

The proximal end of the access lumens 100A of the catheter 100 is positioned at the proximal end of the lumen 112, and its outflow is oriented in the longitudinal direction. The proximal end of the access lumen 100B is juxtaposed to a drainage eye 120, and its outflow is oriented in the longitudinal direction. The proximal end of the access lumen 100C is located adjacent to the proximal end of the lumen 112, and its outflow is oriented transversely outwardly away from the lumen 112.

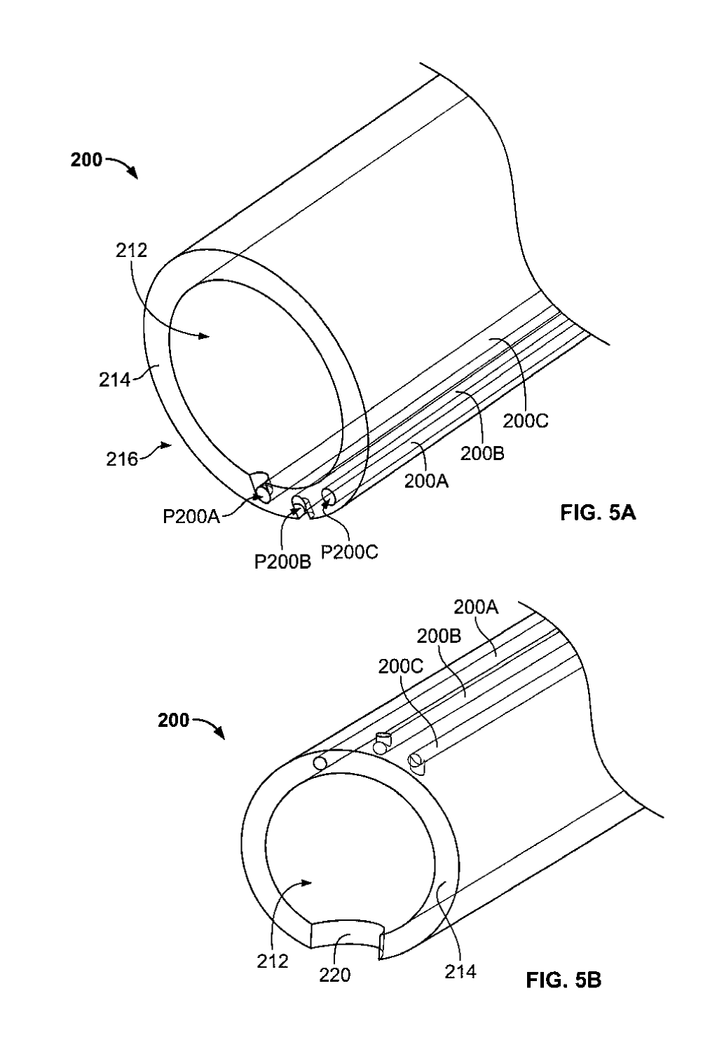

FIGS. 5A and 5B depict a third embodiment of the present invention. Elements illustrated in FIGS. 5A and 5B, which correspond, either identically or substantially, to the elements described above with respect to the embodiment of FIGS. 1-3 have been designated by corresponding reference numerals increased by two hundred. Unless otherwise stated, the embodiments of FIGS. 5A and 5B are constructed and assembled in the same basic manner as the embodiments of FIGS. 1-3.

FIGS. 5A and 5B illustrate a multi-lumen thoracic catheter 200 constructed in accordance with one embodiment of the present invention. The catheter 200 includes a main drainage lumen 212 which is surrounded by a wall 214. According to one embodiment, access lumens 200A, 200B, and 200C are positioned in the wall 214 of the lumen 212.

The proximal ends of the access lumens 200A, 200B, and 200C of the catheter 100 are positioned at the proximal end of the lumen 212. Referring to FIG. 5A, the access lumen 200A has its outflow oriented transversely inwardly into the lumen 212, the access lumen 200B has its outflow oriented transversely outwardly away from the lumen 212, and the access lumen 200C has its outflow oriented in the longitudinal direction. Referring to FIG. 5B, the access lumen 200A has its outflow oriented in the longitudinal direction relative to the lumen 212, the access lumen 200B has its outflow oriented transversely outwardly away from the lumen 212, and the access lumen 200C has its outflow oriented inwardly into the lumen 212. As shown in FIG. 5B, the access lumens 200A, 200B, 200C are positioned opposite diametrically from a drainage space 220.

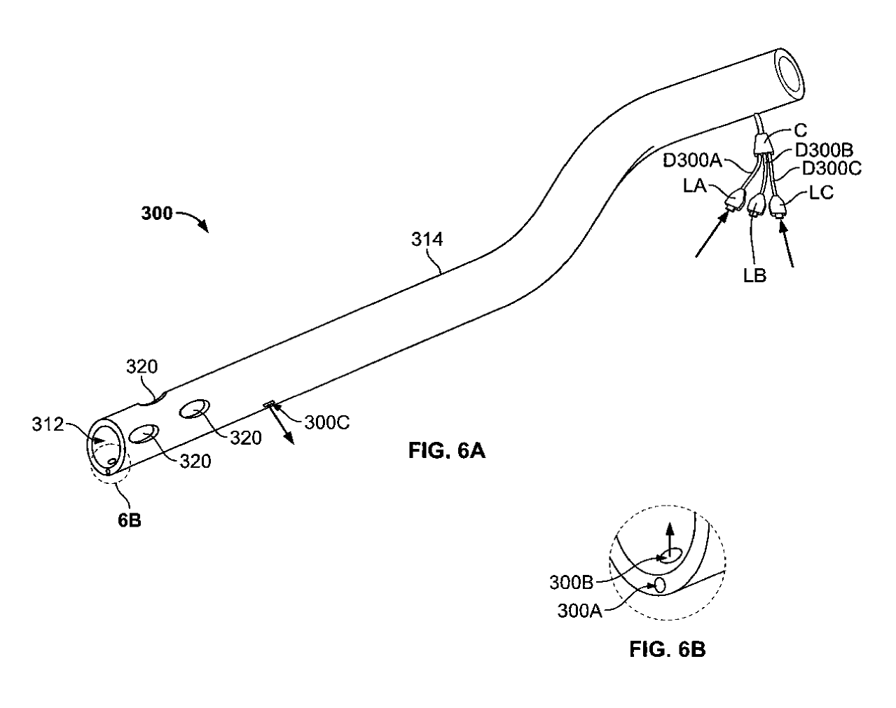

FIGS. 6A and 6B depict a fourth embodiment of the present invention. Elements illustrated in FIGS. 6A and 6B, which correspond, either identically or substantially, to the elements described above with respect to the embodiment of FIGS. 1-3 have been designated by corresponding reference numerals increased by three hundred. Unless otherwise stated, the embodiments of FIGS. 6A and 6B are constructed and assembled in the same basic manner as are the embodiments of FIGS. 1-3.

FIGS. 6A and 6B illustrate a multi-lumen thoracic catheter 300 constructed in accordance with one embodiment of the present invention. The catheter 300 includes a main drainage lumen 312, which is surrounded by a wall 314. According to one embodiment, access lumens 300A, 300B, and 300C are positioned in the wall 314 of the lumen 312. Access lumens 300A, 300B, and 300C have distal ends D300A, D300B, and D300C, respectively. The distal ends D300A, D300B, and D300C are attached to luer-lock valves LA, LB, and LC that are bundled with a clip C.

The proximal ends of access lumens 300A and 300B of the catheter 300 are positioned at the proximal end of the lumen 312. The proximal end of the access lumen 300C is positioned adjacent to the proximal end of the lumen 312, and has its outflow is oriented transversely outwardly away from the lumen 312. The access lumen 300A has its outflow oriented in the longitudinal direction. The access lumen 300B has its outflow oriented transversely inwardly into the lumen 312.

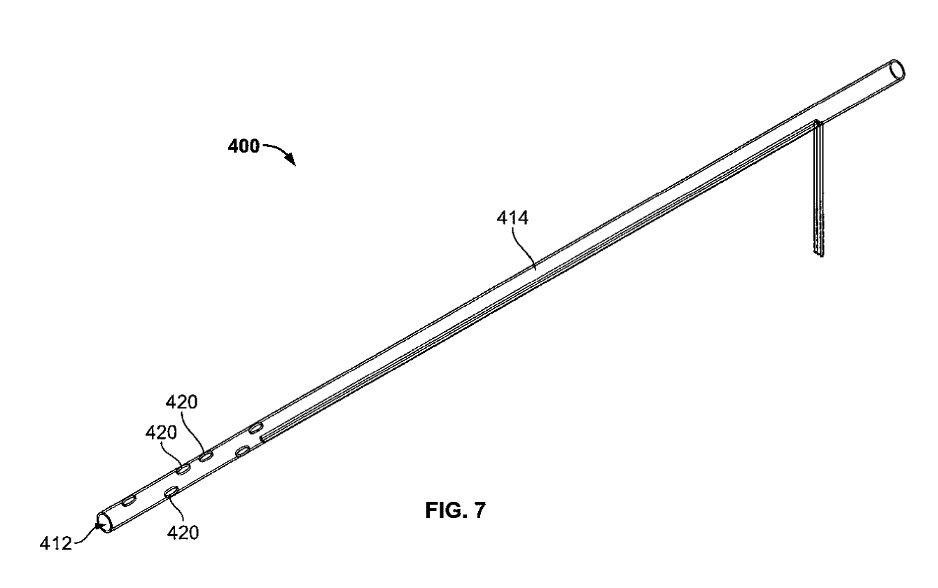

FIGS. 7 to 9 depict a fifth embodiment of the present invention. Elements illustrated in FIGS. 7 to 9, which correspond, either identically or substantially, to the elements described above with respect to the embodiment of FIGS. 1 to 3 have been designated by corresponding reference numerals increased by four hundred. Unless otherwise stated, the embodiments of FIGS. 7 to 9 are constructed and assembled in the same basic manner as the embodiments of FIGS. 1 to 3.

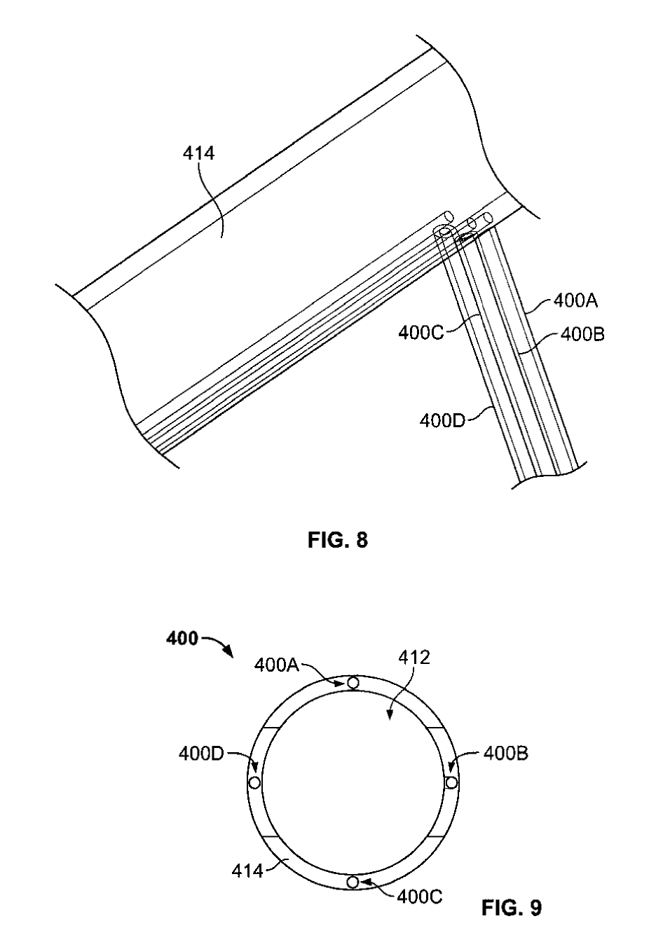

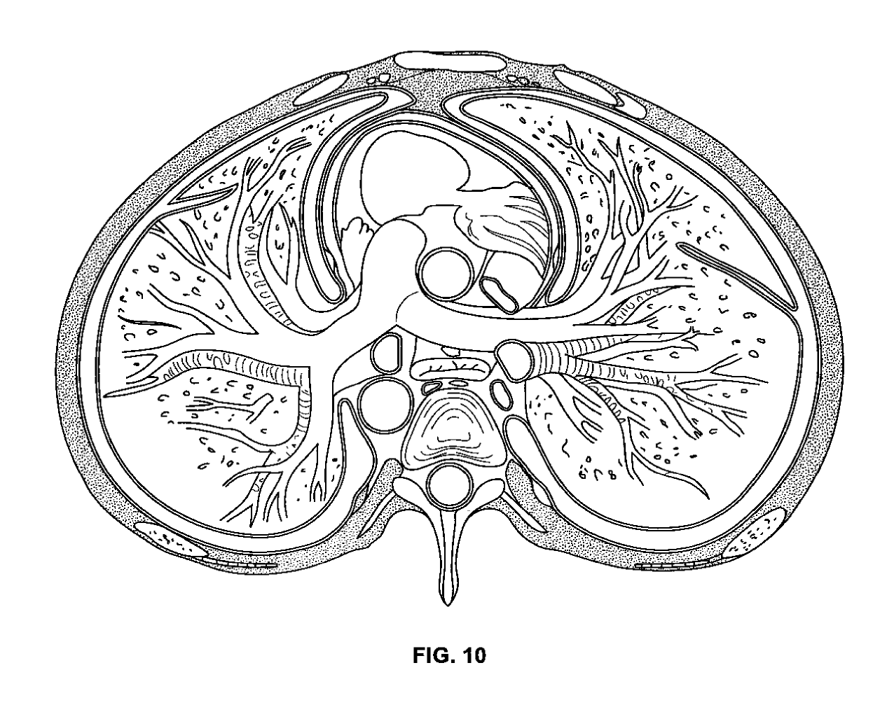

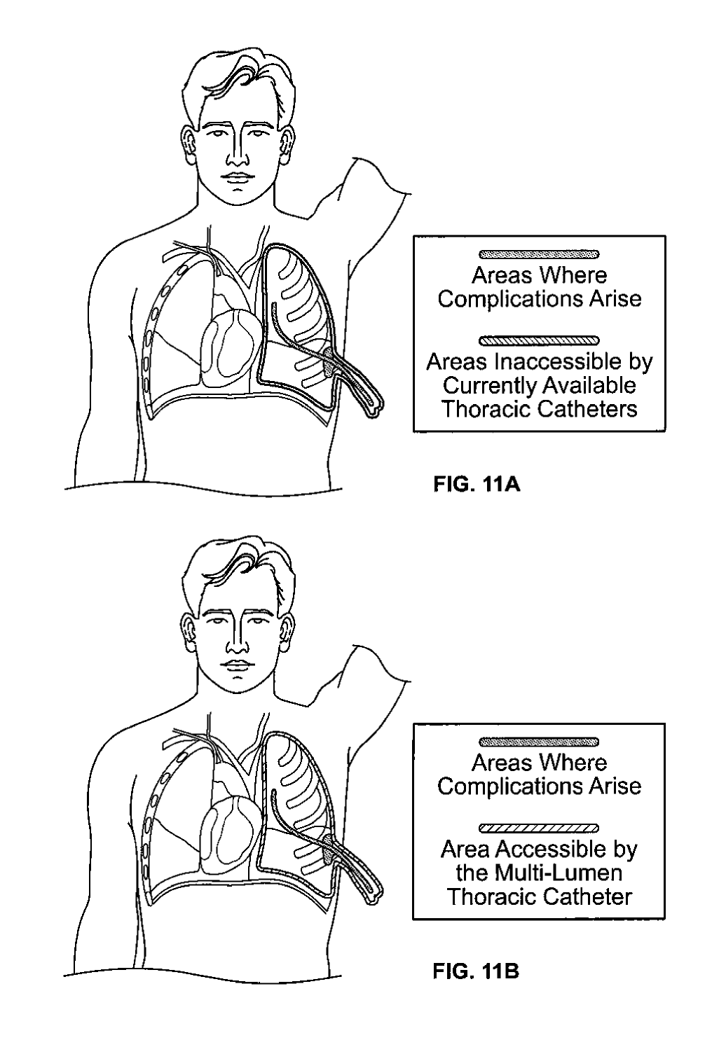

FIGS. 7 to 9 illustrate a multi-lumen thoracic catheter 400 constructed in accordance with one embodiment of the present invention. The catheter 400 includes a main drainage lumen 412 which is surrounded by a wall 414. According to one embodiment, access lumens 400A, 400B, 400C, and 400D are positioned in the wall 414 of the lumen 412 proximal to a plurality of drainage eyes 420, as shown in FIGS. 7 and 8. In an embodiment shown in FIG. 9, the access lumens 400A, 400B, 400C, and 400D are positioned circumferentially within the wall 414 and are spaced apart from one another equidistantly.

It should be appreciated that the present invention provides numerous advantages. For instance, the catheter provides facilities for a medical practitioner to overcome many of the short comings associated with the use of a conventional thoracic catheters (i.e. the dangerous build-up of osculation's in the drainage tube, and pain inflicted on the patient by the drainage tube itself). The catheter provides medical practitioners with a means through which they can access spaces that had been previously inaccessible by conventional thoracic catheters. Thoracic catheters are used in a closed negative pressure system with a suction drainage device. This means that a seal must be maintained between the atmosphere and the skin that contacts the catheter in order to support respiratory functioning, and prevent infection. Because of this, surgeons are not able to access the pleural space or the interior of the main drainage lumen of the thoracic catheter without breaking the seal. This causes a problem when complications arise. The major complications, including occlusion and pain, occur within the regions that are inaccessible by conventional thoracic catheters. The catheter provides access to the pleural space and the interior of the main drainage lumen: it gives physicians the ability to access the areas where complications arise and allows them to actively provide solutions to the underlying causes of the complications.

It should be noted that the present invention can have numerous modifications and variations. For instance the catheter may be provided with only one access lumen which may have openings at periodic intervals along its length. This may facilitate: i) maximizing the internal diameter of the main drainage tube, thus increasing the maximum flow rate through the catheter, or ii) decreasing the outside diameter of the main drainage tube which may contribute to reducing pain in the patient. In addition, the access lumens may be positioned exterior or interior surfaces of the main drainage lumen. Further, the dimensions and configurations of the elements of the catheter 12 may vary according to use and needs. According to one embodiment, a mechanical device (e.g., a bottle brush) is passed through an access lumen that opens into the main drainage lumen, which can be manipulated into the main drainage lumen to clear occlusion.

It will be understood that the embodiment described herein is merely exemplary and that a person skilled in the art may make many variations and modifications without departing from the spirit and scope of the invention. For instance, all such variations and modifications, in addition to those described above, are intended to be included within the scope of the invention as defined in the appended claims.

### 3. Method for Treating or Preventing Fluid or Air Accumulation in a Pleural Space

According to another aspect, the described invention provides a method for treating or preventing fluid accumulation or air accumulation in a body cavity of a subject, the method comprising the steps of:

(a) aseptically inserting through an incision at an insertion site of the subject a multi-lumen catheter comprising a main drainage lumen surrounded by a wall and at least one access lumen positioned in or on the wall;

(b) securing the inserted multi-lumen catheter by closing the incision with a suture;

(c) infusing a physiological solution through at least one access lumen of the multi-lumen catheter to dilute a drainage fluid;

(d) connecting a distal end of a main drainage lumen of the multi-lumen catheter to a suction drainage system; and

(e) applying a vacuum force to the suction drainage system to remove the diluted drainage fluid.

According to one embodiment of the method, the body cavity is a pleural cavity. According to one such embodiment, the body cavity is a cranial cavity. According to another embodiment, the body cavity is a spinal cavity. According to another embodiment, the body cavity is a abdominal cavity. According to another embodiment, the body cavity is a pelvic cavity. According to another embodiment, the physiological solution in (c) comprises a saline solution, Ringer's solution, 5% dextrose in water (D5W), or a mixture thereof.

The multi-lumen thoracic catheter of the describe invention can overcome common complications associated with thoracic catheters. Tube occlusion is prevented by infusing a physiologic solution through the access lumen, which terminates to the interior of the main drainage lumen. The saline mixes with the drainage fluid, diluting it and preventing stagnation, leading to no occlusion. If clotting persists, anti-coagulants can be infused to compliment the saline irrigation. According to another embodiment, tube occlusion is prevented by passing a mechanical device through the lumen that opens into the main drainage lumen, which can be manipulated into the main drainage lumen to clear occlusion. According to one such embodiment, the mechanical device is a bottle brush. According to another embodiment, a thrombolytic agent is infused through the lumen that exits into the main drainage lumen if a clot is present in the tube itself.

According to another embodiment of the method, the suction drainage system includes a three bottle system. The first chamber is the collection chamber, which is directly attached to the thoracic catheter in the subject. Its function is to collect the drained fluid allowing for visualization and recording of the fluid. The second chamber is a water seal chamber. The water seal acts as a one way valve, which prevents air and fluid from returning to the pleural space during inspiration, but allows it to exit otherwise. The water seal can be replaced by a mechanical one way valve used in new dray system models. The third chamber is the suction control chamber, which controls the amount of suction allowed by the system. Maintaining a closed negative pressure system is important in order to prevent lung collapse and to prevent pathogens from entering into the body. According to another embodiment, the suction drainage system is a single-flow drainage system that only allows one direction of flow. Any condition that causes fluid or air accumulation in the pleural space requires suction thoracic catheter insertion to ensure negative pleural pressure, prevent lung collapse, and ensure efficient oxygen delivery. According to some such embodiments, the condition is pneumothorax (a collapsed lung) produced by collection of air in the space around the lungs. The build up of air puts pressure on the lung, so it cannot expand as much as it normally does.

According to some such embodiments, the condition is pleural effusion, meaning an abnormal buildup of fluid between the layers of tissue that line the lungs and chest cavity. The body produces pleural fluid in small amounts to lubricate the surfaces of the pleura, the thin tissue that lines the chest cavity and surrounds the lungs. A pleural effusion is an abnormal, excessive collection of this fluid. There are two different types of effusions that can develop. Transudative pleural effusions are caused by fluid leaking into the pleural space. This is caused by increased pressure in, or low protein content in, the blood vessels. Congestive heart failure is the most common cause. Exudative effusions are caused by blood clots in the lung blood vessels (pulmonary emboli), infection, inflammation, lung injury, and drug reactions.

According to some such embodiments, the condition is chylothorax, a lymphatic fluid accumulation in the pleural space. According to some such embodiments, the condition is empyema, a collection of pus (viscous, yellowish-white fluid formed in infected tissue, consisting of white blood cells, cellular debris, and necrotic tissue) in the space between the lung and the inner surface of the chest wall (pleural space).

According to some such embodiments, the condition is pyogenic infection, meaning an infection characterized by severe local inflammation, usually with pus formation, generally caused by one of the pyogenic bacteria. According to some such embodiments, the condition is hemothorax, meaning the collection of blood in the space between the chest wall and the lung (the pleural cavity). According to some such embodiments, the condition is hydrothorax, meaning accumulation of serous fluid in the pleural space.

Each of these conditions can be a result of a pulmonary disease, a lung infection, a lung cancer, a breast cancer, or any surgery or trauma that affects a negative pressure in the pleural space. (“Pleural Effusion: MedlinePlus Medical Encyclopedia,” National Library of Medicine—National Institutes of Health. Web. Oct. 29, 2010, incorporated by reference herein in its entirety).

Examples of pulmonary diseases that can causes fluid or air accumulation in the pleural space include, but are not limited to, asthma, chronic obstructive pulmonary disease (COPD), lung infections (such as influenza, pneumonia and tuberculosis), and a lung cancer. According to another embodiment, the insertion site is determined by reviewing clinical signs and chest imaging of the subject.

According to another embodiment, the chest imaging comprises chest X-ray, chest fluoroscopy, computed tomography (CT), high-resolution computed tomography (CT), helical (spiral) computed tomography (CT), computed tomography (CT) angiography, magnetic resonance imaging (MRI), or ultrasonography.

Fluoroscopy is a study of moving body structures—similar to an x-ray “movie.” A continuous x-ray beam is passed through the body part being examined. The beam is transmitted to a TV-like monitor so that the body part and its motion can be seen in detail. Fluoroscopy, as an imaging tool, enables physicians to look at many body systems, including the skeletal, digestive, urinary, respiratory, and reproductive systems.

Chest fluoroscopy is a type of x-ray procedure used to assess the motion and function of the lungs and other structures of the respiratory tract. Chest fluoroscopy may be performed when the motion of the lungs, diaphragm (dome-shaped muscle that separates the abdominal cavity from the chest cavity), or other structures in the chest need to be evaluated.

Other related procedures that may be used to diagnose problems of the lungs and respiratory tract include, without limitation, bronchoscopy, computed tomography (CT scan) of the chest, chest x-ray, chest ultrasound, lung biopsy, lung scan, mediastinoscopy, oximetry, peak flow measurement, positron emission tomography (PET) scan, pleural biopsy, pulmonary angiography, pulmonary function tests, and thoracentesis.

According to another embodiment, the area that comprises and surrounds the insertion site is the triangle bordered by the anterior border of the latissimus dorsi, the lateral border of the pectoralis major muscle, a line superior to the horizontal level of the nipple, and an apex below the axilla.

According to another embodiment, the area that comprises and surrounds the insertion site is a lateral thorax, at a line drawn from an armpit to the nipple in male or to the side above the sternoxiphoid junction (lower junction of the sternum (a long flat bone in most vertebrates that is situated along the ventral midline of the thorax and articulates with the ribs)) in female.

According to another embodiment, the size of the incision for the insertion of the multi-lumen thoracic catheter is similar to the diameter of the multi-lumen thoracic catheter being inserted. According to some embodiments, the method further comprises infusing a therapeutic agent through at least one of the access lumen of the multi-lumen thoracic catheter.

According to another embodiment, optionally, pain associated with tissue irritation or tube insertion is reduced by infusing a local anesthetic through an access lumen, which terminates to the exterior of the catheter into the pleural space. The anesthetic will diffuse through all parts of the pleural space, including the insertion site, where it acts on the lacerated parietal pleura. Examples of local anesthetic agents that are suitable for use in the context of the present invention include, but are not limited to, pharmaceutically acceptable salts of lidocaine, benzocaine, bupivacaine, chlorprocaine, dibucaine, etidocaine, marcaine, mepivacaine, tetracaine, dyclonine, hexylcaine, procaine, cocaine, ketamine, pramoxine, and phenol. According to another embodiment, infusion of a local anesthetic, even into the pleural space, is performed while maintaining a low level of suction on the evacuation channel. According to another embodiment, an anti-coagulant is administered through one of the access lumen of the multi-lumen thoracic catheter in order to dissolve blood clots.

Blood clots consist of a plug of platelets in a network of fibrin molecules, and require the enzyme thrombin, calcium ions, and a number of clotting factors to form. Platelets play an essential role in adhering to damaged cells, collagen, and foreign materials and releasing factors which recruit other clotting molecules. Blood normally contains 150,000 to 400,000 platelets per microliter, and if this concentration were to be diluted to 50,000 platelets per microliter, coagulopathy would ensue. Another way of inducing coagulopathy is preventing the binding of the platelets to the surface of the foreign material. This can be accomplished by providing a hydrophilic coating to the material. The hydrophilic coating has a higher affinity for water than for the blood proteins. The bound water then blocks the blood proteins from bonding decreasing clotting.

According to some such embodiments, infusion through any one of the access lumens can be performed either as a bolus (single injection) or as a continuous infusion. A skilled artisan would be able to determine an appropriate flow rate for the infusion of the therapeutic agent, based on the drug being used, the concentration of the drug, the diameter of the access lumen, and the purpose of the infusion.

The algorithm of flow rate and timing of infusion and how it relates to increasing, decreasing or discontinuing the suction through the main evacuation channel of the multi-lumen thoracic catheter can be determined based on the purpose, concentration, substance, and flow rate of the infusion.

Appropriate positioning of the tube, adjustment of flow rate and the vacuum force can allow distribution of therapeutic agent throughout the pleural space of fluids without compressing the lung tissue or causing immediate evacuation of the material without effective distribution throughout the pleural space.

According to one embodiment, the flow rate of infusion ranges from about 1 cc per hour to about 500 cc per hour. Ranges, in various aspects, are expressed herein as from “about” or “approximately” one particular value and/or to “about” or “approximately” another particular value. When values are expressed as approximations by use of the antecedent “about,” it will be understood that some amount of variation is included in the range.

According to another embodiment, the flow rate of infusion ranges from about 1 cc per hour to about 10 cc per hour. According to another embodiment, the flow rate of infusion ranges from about 10 cc per hour to about 20 cc per hour. According to another embodiment, the flow rate of infusion ranges from about 30 cc per hour to about 40 cc per hour. According to another embodiment, the flow rate of infusion ranges from about 40 cc per hour to about 50 cc per hour. According to another embodiment, the flow rate of infusion ranges from about 50 cc per hour to about 60 cc per hour. According to another embodiment, the flow rate of infusion ranges from about 60 cc per hour to about 70 cc per hour. According to another embodiment, the flow rate of infusion ranges from about 70 cc per hour to about 80 cc per hour. According to another embodiment, the flow rate of infusion ranges from about 80 cc per hour to about 90 cc per hour. According to another embodiment, the flow rate of infusion ranges from about 90 cc per hour to about 100 cc per hour. According to another embodiment, the flow rate of infusion ranges from about 100 cc per hour to about 150 cc per hour. According to another embodiment, the flow rate of infusion ranges from about 150 cc per hour to about 200 cc per hour. According to another embodiment, the flow rate of infusion ranges from about 200 cc per hour to about 250 cc per hour. According to another embodiment, the flow rate of infusion ranges from about 250 cc per hour to about 300 cc per hour. According to another embodiment, the flow rate of infusion ranges from about 300 cc per hour to about 350 cc per hour. According to another embodiment, the flow rate of infusion ranges from about 350 cc per hour to about 400 cc per hour. According to another embodiment, the flow rate of infusion ranges from about 400 cc per hour to about 450 cc per hour. According to another embodiment, the flow rate of infusion ranges from about 450 cc per hour to about 500 cc per hour. According to another embodiment, the flow rate of infusion ranges from about 10 cc per hour to about 500 cc per hour. According to another embodiment, the flow rate of infusion ranges from about 50 cc per hour to about 500 cc per hour. According to another embodiment, the flow rate of infusion ranges from about 100 cc per hour to about 500 cc per hour. According to another embodiment, the flow rate of infusion ranges from about 150 cc per hour to about 500 cc per hour. According to another embodiment, the flow rate of infusion ranges from about 200 cc per hour to about 500 cc per hour. According to another embodiment, the flow rate of infusion ranges from about 250 cc per hour to about 500 cc per hour. According to another embodiment, the flow rate of infusion ranges from about 300 cc per hour to about 500 cc per hour. According to another embodiment, the flow rate of infusion ranges from about 350 cc per hour to about 500 cc per hour.

According to another embodiment, the therapeutic agent is an anti-infective agent including, but not limited to, an antibiotic agent, an anti-fungal agent, or antiviral agent. According to some such embodiments, the anti-infective agent treats or prevents a localized infection.

According to another embodiment, the therapeutic agent is a sclerotic agent, which induces adhesion between the parietal and visceral pleura. Suitable sclerotic agents for use in the context of the present invention include, but are not limited to, bleomycin and talc.

According another embodiment, the sclerotic agent is infused as a bolus (single) injection and the vacuum force is discontinued during infusion of the sclerotic agent. According to another embodiment, the sclerotic agent is infused as a bolus (single) injection and the vacuum force is discontinued for one hour.

According to one embodiment, a sclerotic agent is infused as a bolus infusion and the suction of the evacuation channel discontinued for a period of one hour. A sclerotic agent is a compound that acts by irritation of the veinous intimal epithelium. The suction cam then be reinstituted, both to maintain negative pressure in the pleural space and to evacuate any remaining sclerotic agent.

According to another embodiment, the therapeutic agent is an anti-inflammatory agent, which decreases inflammation in the pleural space.

According to another embodiment, the anti-inflammatory agent is a steroidal anti-inflammatory agent. The term “steroidal anti-inflammatory agent” as used herein refer to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, fiucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

According to another embodiment, the anti-inflammatory agent is a non-steroidal anti-inflammatory agent. The term “non-steroidal anti-inflammatory agents” as used herein refers to a large group of agents that are aspirin-like in their action, including ibuprofen (Advil®), naproxen sodium (Aleve®), and acetaminophen (Tylenol®). Additional examples of non-steroidal anti-inflammatory agents that are usable in the context of the present invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

According to another embodiment, the therapeutic agent is a thrombolytic agent, which dissolves clotted blood in the pleural space. The term “thrombolytic agent” is meant to refer to any agent effective in helping to dissolving or breaking up an occluding thrombus. Examples of thrombolytic agents suitable for use in the context of the present invention include, but are not limited to, streptokinase, urokinase, prourokinase, alteplase, reteplase, anistreplase and tissue plasminogen activator (t-PA) and biologically active variants thereof. A combination of two or several thrombolytic agents may be also used.

### 4. Methods for Examining a Pleural Tissue Using Multiple-Lumen Thoracic Catheter

According to another aspect, the described invention provides a method for examining a tissue in a body cavity of a subject, comprising the steps of:

(a) aseptically inserting through an incision at an insertion site of the subject a multi-lumen catheter comprising a main drainage lumen surrounded by a wall and at least one access lumen positioned in or on the wall;

(b) securing the inserted multi-lumen catheter by closing the incision with a suture;

(c) inserting an endoscope through art access lumen of the multi-lumen catheter; and

(d) examining the tissue in the body cavity of the subject.

According to one embodiment of the method, the body cavity is a pleural cavity. According to one such embodiment, the body cavity is a cranial cavity. According to another embodiment, the body cavity is a spinal cavity. According to another embodiment, the body cavity is a abdominal cavity. According to another embodiment, the body cavity is a pelvic cavity.

The term “endoscopy” as used herein refers to a minimally invasive diagnostic medical procedure using an endoscope, which is used to examine the interior surfaces of an organ or tissue. The term “endoscope” used herein refers to a medical device consisting of a long, thin, flexible (or rigid) tube that has a light and a video camera. Images of the inside of the patient's body can be seen on a screen. The whole endoscopy is recorded so that doctors can check it again. The endoscope can also be used for enabling biopsies and for retrieving foreign objects. Endoscopy is a noninvasive alternative to surgery for foreign object removal from the gastrointestinal tract.

According to another embodiment, the method further comprises sampling a tissue in the pleural space, wherein the endoscope comprises endoscopic forceps for tissue biopsy.

According to another embodiment, the method further comprises sampling a tissue in the pleural space, wherein a flexible biopsy forceps that is not incorporated into an endoscope is guided into the body cavity under fluoroscopy or blindly.

According to another embodiment, the method further comprises sampling a tissue in the pleural space, wherein the endoscope comprises endoscopic forceps for tissue biopsy.

### 5. Method for Monitoring the Physical or Biochemical State of Pleural Tissue

According to another aspect, the described invention provides a method for monitoring a physical or biochemical state of a tissue within a body cavity using the multi-lumen catheter, the method comprising the steps of:

(a) aseptically inserting through an incision at an insertion site a multi-lumen catheter comprising a main drainage lumen surrounded by a wall and at least one access lumen positioned in or on the wall;

(b) securing the inserted multi-lumen catheter by closing the incision with a suture;

(c) introducing an instrument that measures the physical or biochemical state of the tissue within the body cavity through at least one access lumen of the multi-lumen catheter; and

(d) monitoring the physical or biochemical state of the tissue within the body cavity, wherein the physical or biochemical state comprises an electrical parameter, a thermal parameter, a photoelectric parameter, a barometric parameter, or a combination thereof.

According to one embodiment of the method, the body cavity is a pleural cavity. According to one such embodiment, the body cavity is a cranial cavity. According to another embodiment, the body cavity is a spinal cavity. According to another embodiment, the body cavity is a abdominal cavity. According to another embodiment, the body cavity is a pelvic cavity.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

The described invention may be embodied in other specific forms without departing from the spirit or essential attributes thereof and, accordingly, reference should be made to the appended claims, rather than to the foregoing specification, as indicating the scope of the invention.

### 5. Examples

The following examples are set forth so as to provide those of ordinary skill in the art with a complete disclosure and description of how to make and use the present invention, and are not intended to limit the scope of the invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Drain Testing Protocol

1.1 Rationale

A drain test is used to examine whether a multi-lumen thoracic catheter of the present invention is proficient in draining various materials from a simulated pleural space while avoiding clot formation within the catheter. The drain test also can test the efficacy of the catheter's drainage ability with a viscous substance, meant to simulate the materials that will need to be evacuated from the pleural space. The end result of the test is the appropriate saline flow rate into the main drainage tube to prevent tube occlusion.

1.2 Introduction

Any condition that causes fluid or air accumulation in the pleural space requires suction thoracic catheter insertion to ensure negative pleural pressure, prevent lung collapse, and to ensure efficient oxygen delivery. Tube occlusion often occurs when the blood's platelets bind to the surface of the catheter's inner diameter. These platelets form a plug, and along with a network of fibrin molecules, the enzyme thrombin, calcium ions, and a number of clotting factors, a blood clot is formed inside the catheter. The multi-lumen thoracic catheter of the described invention can resolve the problem of tube occlusion using saline and the concept of dilutional coagulopathy. By reducing the occurrence of tube occlusion, patient's chest tubes do not have to be changed and their hospital stay and healing time can be minimized.

1.3 Materials

The test is performed using a pulmonary model. The materials needed for the pulmonary model are balloons, a 3 Liter plastic container, 20 gauge tubing, infusion pump, Pleur-evac® Sahara, tubing to connect the vacuum source to the Pleur-evac®, stop cocks, barbed leur lock adapter, large syringe, and super glue.

TABLE 2
Pulmonary Model Materials

| Name | Item Description | Vendor |
|---|---|---|
| Pleur-evac | | Donated by |
| | | Dr. Pearlstone |
| Stop Cock | Polycarbonate Luer 4-way Stopcock | Cole-Palmer |
| Vacuum Gauge | Dixon Valve Steel Lower Mount | Amazon |
| Balloon | Standard Linen White Latex | Bargain Balloons |
| Poland Spring | 3 Liter Plastic Bottle | Fresh Ideas Kings |
| Original, | | |
| 3 Liter | | |
| Caulk | Dap Aquarium Silicone Sealant | Ace Hardware |
| Krazy Glue | Krazy Glue | Ace Hardware |
| Syringe | BD Disposable 30 mL Syringe | Cole-Palmer |
| | (luer-lock Tip) | |

The materials needed for the drain test are the catheter tube, the lumen tube, saline, a mixture of cornstarch and water (simulates blood plasma), eggs, and Jell-O. For testing purposes a 32 French catheter is used, wherein the lumen tube has a diameter of 0.6 mm. Only one of the catheter and lumen tubes is needed for this test as they can be reused for each test run.

TABLE 3
Drain Test Materials

| Name | Item Description | Vendor |
|---|---|---|
| Thoracic Catheter | 28 French Thoracic Catheter | Donated by Teleflex |
| Small Lumen | Genex GT25 20 Gauge | Genex |
| | Clear Plastic Tubing | |
| Saline Bag (IV | Saline 0.9% Sodium Chloride | Donated by Dr. Ritter |
| Injection) | Isotonic Solution | |
| Cornstarch | Cornstarch | Fresh Ideas Kings |
| Eggs | ShopRite Large Eggs | Fresh Ideas Kings |
| Jell-O | Strawberry Gelatin | Fresh Ideas Kings |

1.4 Methods

1.4.1 Test Set-Up

The model consists of a 3 liter bottle with the bottom cut off. This bottle serves to model the chest wall. Two balloons are used in the model to model the lungs and diaphragm. The lung balloon is fitted around the top opening of the bottle and is open to the atmosphere. The diaphragm balloon is placed on the bottom of the bottle and is closed to the atmosphere. This bottle and balloon combination mimics the lung, chest wall, diaphragm, and pleural space. When the diaphragm balloon is pulled downward, inspiration is modeled. The thoracic cavity increases in diameter, lowering the pressure in the pleural space and causing the lung balloon (at atmospheric pressure) to inflate. As the diaphragm balloon returns to normal, expiration is modeled. The thoracic volume is reduced and the pressure rises, causing the lung balloon to deflate. Because of this action, the model simulates the passive lung expansion due to diaphragm movement. A stopcock is placed on the tube between the syringe and the container to regulate the flow rate of the saline. The bottom balloon (simulates diaphragm) is moved manually with a frequency of 0.28 Hz. This action simulates breathing.

1.4.2 Test Protocol

For the drain test, an opening must be made in the container wall to insert the catheter and lumen tubes. The distal end of the catheter is attached to the Pleur-evac®, which in turn is attached to the vacuum source. The distal end of the lumen tube is attached to an infusion pump for the constant injection of saline into the main drainage tube. The infusion pump also regulates the flow rate of the saline being pumped from the lumen tube into the main drainage tube. The flow rate used to irrigate the main drainage tube is tested with a mixture of the three test materials, eggs, mixture of cornstarch and water, and Jell-O. Krazy® glue is also used to secure the catheter and the lumen tubes to the container wall.

1.4.3 Test Material 1: Control

The first test is conducted with a mixture of gelatin, cornstarch and water, and eggs, which is the material within the “pleural space” to be evacuated by the catheter. The mixture of materials includes the following materials: three eggs, 50 ml of gelatin, and fifteen tablespoons of a cornstarch and water mixture. The mixture of materials is measured and placed into the container before the “lung” balloon is secured over the container's mouth. Saline is introduced to the simulated pleural space using the designated lumen and an infusion pump. The saline irrigation lumen is located proximal to the last drainage eye and inserted 1⅛ inches from the container wall. The infusion pump ensures the syringe injects saline into the system at a rate of 1 ml/min, which is the maximum rate at which the body would be producing the materials that will need to be evacuated from the pleural space. The vacuum suction is then initiated using the Pleur-evac® Sahara to control the amount of suction being applied. The Pleur-evac® Sahara should be set to a vacuum setting of about −20 cm H₂O. The test is terminated after 1 hour, or when all the initial material within the simulated pleural space has been completely evacuated, whichever comes first. Once the test is complete, the collection receptacle within the Pleur-evac® Sahara is emptied and made ready for the next test. For this control, no saline is infused directly into the main drainage lumen. This test mimics the actions of the current thoracic catheters.

1.4.4 Test Material 2: Saline Dilution

The next test also consists of placing eggs, gelatin, and cornstarch and water into the “pleural space.” The mixture of materials use the same proportions as the previous test and simulates the viscous and “chunky” nature of the materials that may need to be evacuated from the pleural space. Eggs possess proteins that are similar to blood, and therefore can model whether or not the proteins can adhere to the catheter causing clots to form. The cornstarch and gelatin contribute to the viscosity of the drainage material. The test is conducted with the same procedure as the control experiment, except for the fact that saline is infused through a lumen into the main drainage tube to facilitate drainage. An infusion pump is used to regulate the flow rate of the saline from the lumen tube into the catheter for saline irrigation. The saline flow rate that is tested is 5 ml/min. This test, like the control, is terminated when either one hour is completed, or all of the drainage materials have been evacuated from the pleural space.

Between each test, the interior of the catheter is also checked after each test to determine whether or not a clot has formed. If a clot forms within the catheter during the control tests, the drainage incompetence of the existing catheter is verified. If a clot forms inside the catheter while it is being flushed with saline, it is concluded that saline dilution is not a viable solution to the tube occlusion. If no clot has formed within the catheter while it is being washed with saline, then the efficacy of the drainage system is proved. The efficacy of the multi lumen design is also verified if a clot forms within the catheter and is able to be dislodged and removed using saline dilution.

1.5 Results

Drain Test

During the drain test, observations were made for both a control and using saline dilution. It was observed during the control that after the removal of the majority of the fluid, there was quite a large amount of residual material left in the tube. Over the course of the test, this material was not drained off and remained stagnant in the catheter. During the tests where saline dilution was used, it was observed that no material stagnated in the tube. Any residual material left over in the catheter after the initial removal was seen to slide out of the tube in a short period of time (between 1 and 2 minutes). No material remained in the tube for long periods as was observed during the control. It was also observed that at faster rates of saline dilution, the residual material in the tube was cleaned out more quickly. Finally, it was also seen that a manual “saline wash” could be performed in which a manual high pressure was exerted on the saline dilution tube to more quickly and more effectively remove the residual material in the catheter.

### Example 2

### Dye Movement in Pulmonary Model Testing Protocol

2.1 Rationale

The purpose of this test is to determine whether or not the access lumen is effective in the introduction of fluid into the pleural space. The test examines both infusion and injection methods of fluid injection through the access lumen. The test is designed to test whether or not the injected fluid can successfully diffuse not only to the wound site, but through the entire pleural space. This validates the efficacy of the ability of the access lumen to introduce fluid into the pleural space.

2.2 Introduction

Currently, physicians do not have direct access to the pleural space when a chest tube is placed, nor do they typically invade the area to administer fluid or drugs (such as anesthetic or anticoagulant). However, it is important to have an access to the pleural space, most specifically the pleural wound site. For fluids administered at this location, it is beneficial to have the fluid reach and interact at this wound site. Therefore, whether or not injection or infusion of a fluid can diffuse to the wound site, and the rest of the pleural space was determined.

In a clinical sense, this test allows for validation that the fluid infused or injected through the access lumen allows physicians the most direct access to the pleural wound site for administration of fluids of their choice. For the specific use of anesthetic, the tube allows for the direct administration of local anesthetic to the wound site, reducing the need for systemic anesthetic. It has been observed that the use of infused local anesthetic results in significant pain reduction after surgery.

2.3 Materials

The materials used for the pulmonary model in the drain test are equivalent to the materials needed for the pulmonary model for this testing protocol (see Table 2 above).

TABLE 4
Dye Test Additional Materials

| Name | Item Description | Vendor |
|---|---|---|
| Infusion Pump | | |
| Thoracic Catheter | 28 French Thoracic Catheter | Donated by Teleflex |
| Small Lumen | Genex GT25 20 Gauge Clear | Genex |
| | Plastic Tubing | |
| Saline Bag (IV | Saline 0.9% Sodium | Donated by Dr. |
| Injection) | Chloride Isotonic Solution | Ritter |
| Dye | Food coloring 15 V 0071 | Ward's Natural |
| | | Science |
| Spectrophotometer | SP-830 Digital | |
| | Spectrophotometer - 120 V | |

2.4 Methods

2.4.1 Test Set-Up

This test is designed to determine the concentration of dye (used to mimic anesthetic or other physician input) present in the pleural space. To perform this test, a pulmonary model is needed. There are also a number of accessory items needed in the test set-up.

A syringe is used to continuously infuse extra fluid into the pleural space of the model simulating a pleural effusion. The next two additions to the model for this test are a dye injection tube and syringe, and a sample extraction tubes and syringes. The first sample extraction tube located at the wall of the container is used to monitor the dye concentration that would be at the wound site. The next sample extraction tube, located three inches from the simulated wound site, is utilized to examine the amount of dye diffusion through the pleural space. The dye injection tube is one inch from wall of the container. The final tube entering the model is the Atrium 6 eyed 32 French PVC thoracic catheter, which is attached to a suction system (Pleur-evac® Sahara) and terminate inside the pleural space of the model. All entry points into the bottle must be sealed (for example with Krazy® Glue). Because the pleural space of the model is under the influence of a vacuum, the model must be sealed. For all tubing entering the model, except for the thoracic catheter, a stopcock is attached to the proximal end of the tube. This allows for maintenance of the vacuum in the model and for syringe attachment for injection or sampling of fluid.

2.4.2 Standard Curve

A standard curve of percent concentration vs. absorbance is obtained for the dye. Solutions of dye and saline are made at the following percent volumes:

| Volume | Volume | |
|---|---|---|
| Fraction | Fraction | Percent Dye |
| Dye | Saline | Concentration |
| 1 | 0 | 100% |
| 0.75 | 0.25 | 75% |
| 0.5 | 0.5 | 50% |
| 0.25 | 0.75 | 25% |
| 0 | 1 | 0% |

A Turner Spectrophotometer SP-830 is used to measure the absorbance of each solution at 685 nm. A percent concentration vs. absorbance plot is then made to compare values in the following test.

2.4.3 Test Procedure

The first task in set-up is the placement of the dye injection tube (relative to the distal end of the thoracic catheter and the wall terminus). The dye injection tube is a constant 1 inch from the wall terminus. The sample extraction tubes are placed directly at the wall terminus (simulated wound site and point of anesthetic effect), and at an extreme location on the plastic container 3 inches from the wound site. This additional sample extraction point allows for a more accurate measure of the degree of fluid dissipation within the pleural space.

The model is initially filled with 1000 ml of saline and the lung balloon is inserted and sealed. The suction system is then turned on suction is adjusted to equilibrium. Throughout the duration of the test, 1 ml of additional saline is injected into the system every minute to simulate additional fluid accumulation in the pleural space. The balloon that acts as a diaphragm is manually pulled in and out at a rate of 0.28 Hz (16 breaths/minute) for the duration of the test to simulate the average resting rate of breathing. Also at the beginning of the test the dye is infused into the model using an infusion pump at a rate of 2 cc/hr1. Measurements are taken using the sample extraction tube at the following time intervals: 30 seconds, 1 minute, 2 minutes, 5 minutes, 10 minutes, 30 minutes, and 1 hour. Each measurement consists of the extraction of approximately 2 ml of fluid from the fluid extraction tube. Because the extraction tube is continually filled with fluid, the volume of fluid in the lumen must be disregarded during sample extraction. The test is terminated after the final sample (1 hour) is taken. Each sample extracted from the model is placed in the colorimeter and an absorbance value is determined and recorded. Another test is performed using an injection of the dye into the pleural space instead of being infused. 2 mL of dye is injected into the pleural space at the beginning of the test.

2.4.4 Test Results

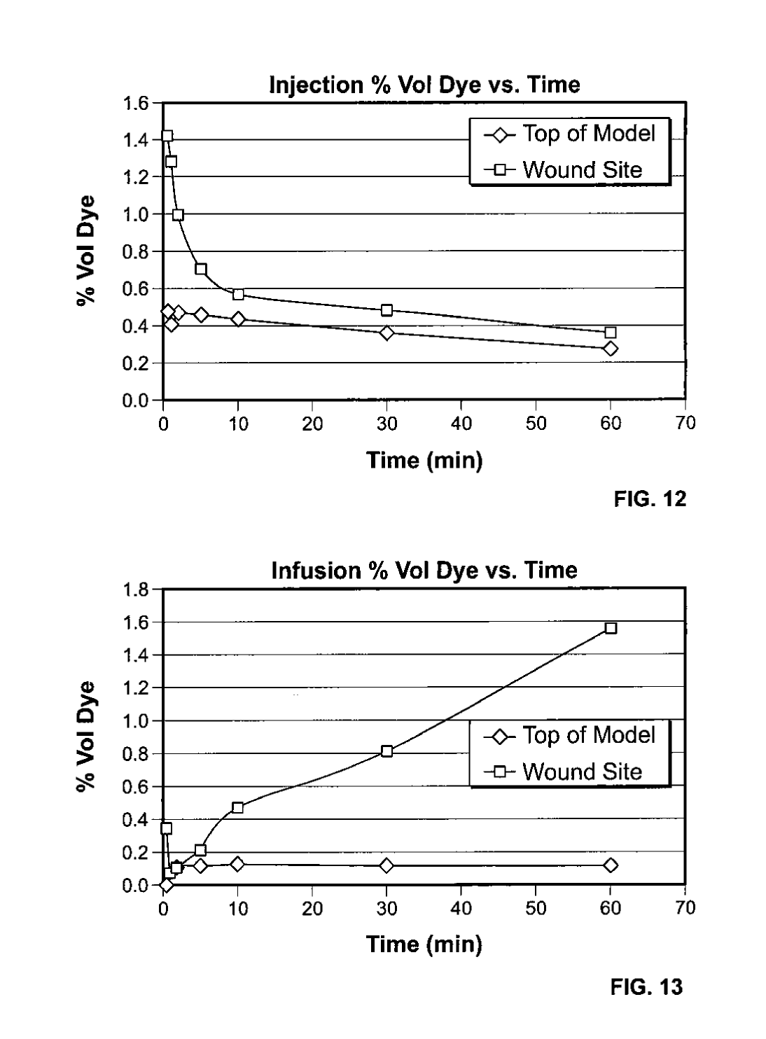

The results of this test are in the form of concentration measurements. Each sample removed from the model (described above) is placed in the colorimeter and an absorbance is recorded. This absorbance value is compared to the generated standard curve and a dye concentration is generated. From these concentration values, a plot of concentration vs. time is generated for each infusion and injection fluid delivery systems. FIG. 12 shows the concentration of dye vs. time plot for injection, as well as a fluid injected through the access lumen can diffuse to the wound site and exist in significant concentrations relative to the rest of the pleural space. FIG. 13 shows the concentration of dye vs. time plot for infusion, as well as a fluid injected through the access lumen can diffuse to the wound site and exist in significant concentrations relative to the rest of the pleural space.

2.4.5 Data Analysis

The data analysis performed for this test is the comparison of the concentration vs. time plots. The goal of this test is to optimize the amount of fluid (dye) that reaches the wound site (wall terminus) and that can successfully diffuse around the pleural space. It is the assumption that in an ideal case, 100% of the fluid should reach the wound site. Therefore, the analysis must be toward which method of fluid delivery, injection or infusion, gives the highest concentrations at the wound site, and which diffuses throughout the entire pleural space. This is determined simply by comparing the plots and determining which fluid delivery method indeed shows the highest concentration of dye over time at the wall terminus, and which shows more diffusion through the pleural space.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

What is claimed is:

1. A multi-lumen catheter for use in a cavity, comprising a main lumen surrounded by a wall; and at least one access lumen positioned in or on the wall, wherein said at least one access lumen conveys a solution to the cavity and said main lumen.

2. A method for treating or preventing fluid accumulation or air accumulation in a body cavity of a subject using the multi-lumen catheter of claim 1, the method comprising:
(a) aseptically inserting through an incision at an insertion site of the subject the multi-lumen catheter comprising a main drainage lumen surrounded by a wall and at least one access lumen positioned in or on the wall;
(b) securing the inserted multi-lumen catheter by closing the incision with a suture;
(c) infusing a physiological solution through at least one access lumen of the multi-lumen catheter to dilute a drainage fluid;
(d) connecting a distal end of a main drainage lumen of the multi-lumen catheter to a suction drainage system; and
(e) applying a vacuum force to the suction drainage system to remove the diluted drainage fluid.

3. The method according to claim 2, wherein the body cavity is a pleural cavity.

4. The method according to claim 2, wherein the body cavity is a cranial cavity.

5. The method according to claim 2, wherein the body cavity is a spinal cavity.

6. The method according to claim 2, wherein the body cavity is a abdominal cavity.

7. The method according to claim 2, wherein the body cavity is a pelvic cavity.

8. The method according to claim 2, wherein the physiological solution in (c) comprises a saline solution, Ringer's solution, 5% dextrose in water (D5W), or a mixture thereof.

9. The method according to claim 2, wherein a thrombolytic agent is infused through an access lumen that exits into the main drainage lumen.

10. The method according to claim 2, wherein the suction drainage system is a single-flow drainage system that only allows one direction of flow.

11. The method according to claim 2, wherein the suction drainage system comprises a collection chamber, a water seal chamber, and a suction control chamber,
wherein the collection chamber attaches the multi-lumen thoracic catheter to the subject;
wherein the water seal chamber prevents air and fluid from returning to the pleural space; during inspiration; and
wherein the suction control chamber controls the amount of suction allowed by the suction drainage system.

12. The method according to claim 3, wherein the fluid accumulation or air accumulation in the pleural cavity of the subject results from a condition comprising pneumothorax, pleural effusion, chylothorax, empyema, hemothorax, hydrothorax, or a combination thereof.

13. The method according to claim 3, wherein the fluid accumulation or air accumulation in the pleural cavity of the subject results from a condition selected from the group consisting of a pulmonary disease, a lung infection, a lung cancer, a breast cancer, and a surgery that affects a negative pressure in the pleural space.

14. The method according to claim 3, wherein the insertion site is determined by reviewing clinical signs and chest imaging of the subject.

15. The method according to claim 3, wherein the chest imaging comprises chest X-ray, chest fluoroscopy, computed tomography (CT), high-resolution computed tomography (CT), helical (spiral) computed tomography (CT), computed tomography (CT) angiography, magnetic resonance imaging (MRI), or ultrasonography.

16. The method according to claim 3, wherein the insertion site is a lateral thorax, at a line drawn from an armpit to the nipple in male or to the side above the sternoxiphoid junction (lower junction of the sternum, or chest bone) in female.

17. The method according to claim 3, wherein a size of the incision for the insertion of the multi-lumen catheter is similar to the diameter of the multi-lumen thoracic catheter being inserted.

18. The method according to claim 2, wherein the method further comprises infusing a therapeutic agent through a second access lumen of the multi-lumen catheter.

19. The method according to claim 18, wherein the therapeutic agent is a local anesthetic agent, and wherein the local anesthetic agent decreases pain associated with tissue irritation or tube insertion.

20. The method according to claim 19, wherein the local anesthetic is selected from the group consisting of benzocaine, lidocaine, and marcaine.

21. The method according to claim 18, wherein the therapeutic agent is an anti-coagulant agent.

22. The method according to claim 18, wherein infusing is performed as a bolus single infusion.

23. The method according to claim 18, wherein infusing is performed as continuous infusion.

24. The method according to claim 18, wherein the therapeutic agent is infused at a flow rate ranging from about 1 cc per hour to about 500 cc per hour.

25. The method according to claim 18, wherein the therapeutic agent is an anti-infective agent comprising an antibiotic agent, an anti-tuberculin agent, an anti-fungal agent, or antiviral agent, wherein the anti-infective agent treats or prevents a localized infection.

26. The method according to claim 18, wherein the therapeutic agent is anti-fungal agent.

27. The method according to claim 18, wherein the therapeutic agent is anti-tuberculin agent.

28. The method according to claim 18, wherein the therapeutic agent is a sclerotic agent, wherein the sclerotic agent induces adhesion between the parietal and visceral pleura.

29. The method according to claim 28, wherein the sclerotic agent is infused as a bolus injection, and wherein the vacuum force is discontinued for an hour.

30. The method according to claim 18, wherein the therapeutic agent is an anti-inflammatory agent, and wherein the anti-inflammatory agent decreases inflammation in the pleural space.

31. The method according to claim 18, wherein the therapeutic agent is a thrombolytic agent, and wherein the thrombolytic agent dissolves clotted blood in the pleural space.

32. A method for examining a tissue in a body cavity of a subject using the multi-lumen catheter according to claim 1, the method comprising:
(a) aseptically inserting through an incision at an insertion site of the subject the multi-lumen catheter comprising a main drainage lumen surrounded by a wall and at least one access lumen positioned in or on the wall;
(b) securing the inserted multi-lumen catheter by closing the incision with a suture;
(c) inserting an endoscope through an access lumen of the multi-lumen catheter; and
(d) examining the tissue in the body cavity of the subject.

33. The method according to claim 32, wherein the body cavity is a pleural cavity.

34. The method according to claim 32, wherein the body cavity is a cranial cavity.

35. The method according to claim 32, wherein the body cavity is a spinal cavity.

36. The method according to claim 32, wherein the body cavity is a abdominal cavity.

37. The method according to claim 32, wherein the body cavity is a pelvic cavity.

38. The method according to claim 32, further comprising sampling a tissue in the pleural space, wherein the endoscope comprises endoscopic forceps for tissue biopsy.

39. The method according to claim 32, further comprising sampling a tissue in the pleural space, wherein a flexible biopsy forceps that is not incorporated into an endoscope is guided into the body cavity under fluoroscopy or blindly.

40. A method for monitoring a physical or biochemical state of a tissue within a body cavity using the multi-lumen catheter of claim 1, the method comprising:
(a) aseptically inserting through an incision at an insertion site the multi-lumen catheter comprising a main drainage lumen surrounded by a wall and at least one access lumen positioned in or on the wall;
(b) securing the inserted multi-lumen catheter by closing the incision with a suture;
(c) introducing an instrument that measures the physical or biochemical state of the tissue within the body cavity through at least one access lumen of the multi-lumen catheter; and
(d) monitoring the physical or biochemical state of the tissue within the body cavity,
wherein the physical or biochemical state comprises an electrical parameter, a thermal parameter, a photoelectric parameter, a barometric parameter, or a combination thereof.

41. The method according to claim 40, wherein the body cavity is a pleural cavity.

42. The method according to claim 40, wherein the body cavity is a cranial cavity.

43. The method according to claim 40, wherein the body cavity is a spinal cavity.

44. The method according to claim 40, wherein the body cavity is a abdominal cavity.

45. The method according to claim 40, wherein the body cavity is a pelvic cavity.
